(19) United States
(12) Patent Application Publication
(10) Pub. No.: US 2018/0140753 A1
(43) Pub. Date: May 24, 2018

(54) COMPRESSIBLE NEGATIVE PRESSURE SOURCE AND METHODS OF USE

(71) Applicant: SMITH & NEPHEW PLC, London (GB)

(72) Inventors: Ben Alan Askem, East Yorkshire (GB); Stephanie Noble, Hull (GB)

(21) Appl. No.: 15/576,224

(22) PCT Filed: May 23, 2016
(86) PCT No.: PCT/EP2016/061612

§ 371 Date: Nov. 21, 2017

Related U.S. Application Data
(60) Provisional application No. 62/166,472, filed on May 26, 2015.

(51) Int. Cl. A61M 1/00; A61F 13/02

(52) U.S. Cl. CPC A61M 1/0003; A61M 1/0035; A61F 13/0206; A61M 1/0033; A61F 13/0209; A61M 2205/587; A61F 13/0216; A61M 1/0011; A61M 2205/581; A61M 2205/18; A61M 1/0088

(57) ABSTRACT

A system, method, and apparatus are disclosed for the application of negative pressure to a desired location. The apparatus comprises a flexible member comprising a fluid impermeable outer membrane defining an internal cavity and one or more compressible members disposed therein and configured to exert an expansion force within the internal cavity that applies a desired level of negative pressure to the wound.

### BACKGROUND

### Field

Embodiments described herein relate to apparatuses, systems, and methods for the application of negative pressure to a desired location, and in certain embodiments relate to a compressive negative pressure source for use in negative pressure wound therapy.

### Description of the Related Art

The treatment of open or chronic wounds that are too large to spontaneously close or otherwise fail to heal by means of applying negative pressure to the site of the wound is well known in the art. Negative pressure wound therapy (NPWT) systems currently known in the art commonly involve placing a cover that is impermeable or semi-permeable to fluids over the wound, using various means to seal the cover to the tissue of the patient surrounding the wound, and connecting a source of negative pressure (such as a vacuum pump) to the cover in a manner so that negative pressure is created and maintained under the cover. It is believed that such negative pressures promote wound healing by facilitating the formation of granulation tissue at the wound site and assisting the body's normal inflammatory process while simultaneously removing excess fluid, which may contain adverse cytokines and/or bacteria. However, further improvements in NPWT are needed to fully realize the benefits of treatment.

Many different types of wound dressings are known for aiding in NPWT systems. These different types of wound dressings include many different types of materials and layers, for example, gauze, pads, foam pads or multi-layer wound dressings. One example of a multi-layer wound dressing is the PICO dressing, available from Smith & Nephew, which includes a superabsorbent layer beneath a backing layer to provide a canister-less system for treating a wound with NPWT. The wound dressing may be sealed to a suction port providing connection to a length of tubing, which may be used to pump fluid out of the dressing and/or to transmit negative pressure from a pump to the wound dressing.

Prior art apparatuses for the application of negative pressure have been large, heavy, rigid, and difficult to use. Such apparatuses often require batteries or sources of power for operation and can hinder a user's mobility.

### SUMMARY

According to some embodiments there is provided a wound treatment apparatus comprising:
a flexible member configured to apply negative pressure to a wound, comprising:
   a fluid impermeable outer membrane defining an internal cavity;
   one or more compressible members disposed in said internal cavity, wherein the one or more compressible members are configured to compress upon removal of air from the internal cavity;
   wherein after the removal of air from the internal cavity, the one or more compressible members are configured to exert an expansion force within the internal cavity that applies a desired level of negative pressure to the wound.

In some embodiments the flexible member is a wound dressing that further comprises a wound contact layer. The one or more compressible members may be disposed in the internal cavity defined between the fluid impermeable outer membrane and the wound contact layer. The wound dressing may further comprise a superabsorbent layer over the one or more compressible members. In some embodiments the fluid impermeable outer membrane comprises a fluid impermeable upper membrane and a fluid impermeable lower membrane. The upper membrane and lower membrane may be sealed together to form a cavity therebetween. In some embodiments the fluid impermeable outer membrane may comprise a bag defining the internal cavity. In some embodiments the flexible member is configured to be wearable on the body of a user. In some embodiments the wound treatment apparatus may further comprise a separate wound dressing configured to be positioned over a wound. The flexible member may be configured to be in fluid communication with the separate wound dressing and apply negative pressure to the wound through the separate wound dressing. A conduit may be in fluid communication with the internal cavity of the flexible member. The conduit may be configured to communicate negative pressure generated by the expansion force of the one or more compressible members to the separate wound dressing. A regulator valve may regulate the negative pressure supplied to the wound dressing by the internal cavity of the flexible member via the conduit. The internal cavity of the flexible member may apply at least −400 mmHg of pressure to the regulator valve. In some embodiments the one or more compressible members may comprise a plurality of constant tension springs. The one or more compressible members may be incorporated into a 3D knitted or fabric material. The 3D fabric or knitted material may comprise a first fabric layer and a second fabric layer and a plurality of constant tension springs extending therebetween. The 3D fabric or knitted layer may comprise a top textured polyester layer, a bottom flat polyester layer, and a third fiber layer comprising one or more compressible members therebetween. In some embodiments the one or more compressible members comprise foam. In some embodiments the one or more compressible members are configured to exert an expansion force within the internal cavity that applies at least −45 mmHg of pressure to the wound. In some embodiments the apparatus may comprise tubing that is connected to the flexible member. The tubing may provide an air leak to allow air to be removed from the cavity. The apparatus may comprise a valve to regulate the removal of air from the internal cavity. In some embodiments air may be removed from the flexible member by squeezing the internal cavity of the flexible member. In some embodiments air may be removed from the internal cavity of the flexible member by a source of negative pressure. In some embodiments the flexible member may comprise a port in fluid communication with the internal cavity. The port may communicate with a source of negative pressure to remove air from the internal cavity. In some embodiments the apparatus may comprise an audible alarm system. The audible alarm system may sound when the pressure in the internal cavity of the flexible member rises above a predetermined negative pressure.

According to some embodiments there is provided herein a method of generating negative pressure, comprising:
providing a flexible member comprising a fluid impermeable outer membrane defining an internal cavity, and one or more compressible members disposed in the internal cavity; and
removing air from the internal cavity, wherein the removal of air causes the one or more compressible members to compress;
wherein after the removal of air from the internal cavity, the one or more compressible members exert an expansion force within the internal cavity to generate a negative pressure to a desired location.

In some embodiments air is removed from the internal cavity via tubing connected to the flexible member. The tubing may provide an air leak to allow air to be removed from the internal cavity. In some embodiments air is removed from the internal cavity by a source of negative pressure is in fluid communication with the internal cavity. In some embodiments air is removed from the internal cavity by squeezing the flexible member. In some embodiments the one or more compressible members comprises foam. In some embodiments the one or more compressible members may comprise a plurality of constant tension springs. The one or more compressible members may be incorporated into a 3D knitted or fabric material. In some embodiments the removal of air from the internal cavity is regulated with a valve. In some embodiments the pressure in the internal cavity is monitored and an alarm may sound when the pressure in the internal cavity rises above a predetermined negative pressure. In some embodiments the one or more compressible members exert an expansion force within the internal cavity that applies at least −45 mmHg to the desired location. In some embodiments the amount of negative pressure generated at a desired location may be regulated with a regulator valve. The regulator valve may be disposed between the flexible member and the desired location. In some embodiments the one or more compressible members exert an expansion force within the internal cavity that applies at least −400 mmHg to the regulator valve.

### BRIEF DESCRIPTION OF THE DRAWINGS

The invention will be better understood from the Detailed Description and from the appended drawings, which are meant to illustrate and not to limit the invention, and wherein:

FIG. 1A illustrate a cross sectional view of an embodiment of a compressible negative pressure source;

FIG. 1B illustrates a top view of an embodiment of a compressible negative pressure source;

FIG. 1C illustrates an exploded view of an embodiment of a compressible negative pressure source;

FIG. 2A illustrates a top view of an embodiment of a 3D fabric incorporating a plurality of compressible members;

FIG. 2B illustrates a cross sectional view embodiment of a 3D fabric incorporating a plurality of compressible members;

FIG. 2C illustrates a perspective view of an embodiment of a 3D fabric incorporating a plurality of compressible members;

FIG. 3 illustrates an embodiment of a wound treatment apparatus including a compressible negative pressure source and a wound dressing;

FIG. 4A illustrates a top view of an embodiment of a wound dressing comprising a compressible negative pressure source;

FIG. 4B illustrates a cross sectional view of an embodiment of a wound dressing comprising a compressible negative pressure source;

FIG. 4C illustrates an exploded view of an embodiment of a wound dressing comprising a compressible negative pressure source

### DETAILED DESCRIPTION

Embodiments disclosed herein relate to apparatuses and methods of treating a wound with reduced pressure, including pump and wound dressing components and apparatuses. The apparatuses and components comprising the wound overlay and packing materials, if any, are sometimes collectively referred to herein as dressings.

It will be appreciated that throughout this specification reference is made to a wound. It is to be understood that the term wound is to be broadly construed and encompasses open and closed wounds in which skin is torn, cut or punctured or where trauma causes a contusion, or any other superficial or other conditions or imperfections on the skin of a patient or otherwise that benefit from reduced pressure treatment. A wound is thus broadly defined as any damaged region of tissue where fluid may or may not be produced. Examples of such wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, burns, diabetic ulcers, pressure ulcers, stoma, surgical wounds, trauma and venous ulcers or the like.

It will be understood that embodiments of the present disclosure are generally applicable to use in topical negative pressure (“TNP”) therapy systems. Briefly, negative pressure wound therapy assists in the closure and healing of many forms of “hard to heal” wounds by reducing tissue oedema; encouraging blood flow and granular tissue formation; removing excess exudate and may reduce bacterial load (and thus infection risk). In addition, the therapy allows for less disturbance of a wound leading to more rapid healing. TNP therapy systems may also assist on the healing of surgically closed wounds by removing fluid and by helping to stabilize the tissue in the apposed position of closure. A further beneficial use of TNP therapy can be found in grafts and flaps where removal of excess fluid is important and close proximity of the graft to tissue is required in order to ensure tissue viability.

As is used herein, reduced or negative pressure levels, such as −X mmHg, represent pressure levels that are below standard atmospheric pressure, which corresponds to 760 mmHg (or 1 atm, 29.93 inHg, 101.325 kPa, 14.696 psi, etc.). Accordingly, a negative pressure value of −X mmHg reflects absolute pressure that is X mmHg below 760 mmHg or, in other words, an absolute pressure of (760−X) mmHg. In addition, negative pressure that is “less” or “smaller” than X mmHg corresponds to pressure that is closer to atmospheric pressure (e.g., −40 mmHg is less than −60 mmHg). Negative pressure that is “more” or “greater” than −X mmHg corresponds to pressure that is further from atmospheric pressure (e.g., −80 mmHg is more than −60 mmHg).

The negative pressure range for some embodiments of the present disclosure can be between about −20 mmHg and −200 mmHg Note that these pressures are relative to normal ambient atmospheric pressure. Thus, −200 mmHg would be about 560 mmHg in practical terms. In some embodiments, the pressure range can be between about −40 mmHg and −150 mmHg. Alternatively a pressure range of up to −75 mmHg, up to −80 mmHg or over −80 mmHg can be used. Also in other embodiments a pressure range of below −75 mmHg can be used. Alternatively, a pressure range of over approximately −100 mmHg, or even 150 mmHg, can be supplied by the negative pressure apparatus.

Embodiments of the wound dressings, wound treatment apparatuses and methods described herein may also be used in combination or in addition to those described in U.S. Pat. No. 9,061,095, titled “WOUND DRESSING AND METHOD OF USE”; PCT Patent Publication No. WO 2011/144888 A1, titled “WOUND PROTECTION”; and PCT Patent Publication No. WO 2014/020440 A1, titled “WOUND DRESSING,” which are hereby incorporated by reference in their entireties, including further details relating to embodiments of wound dressings, the wound dressing components and principles, and the materials used for the wound dressings.

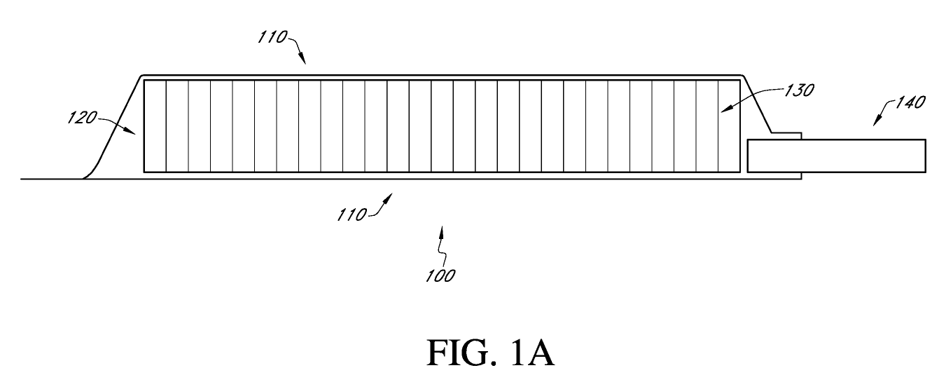

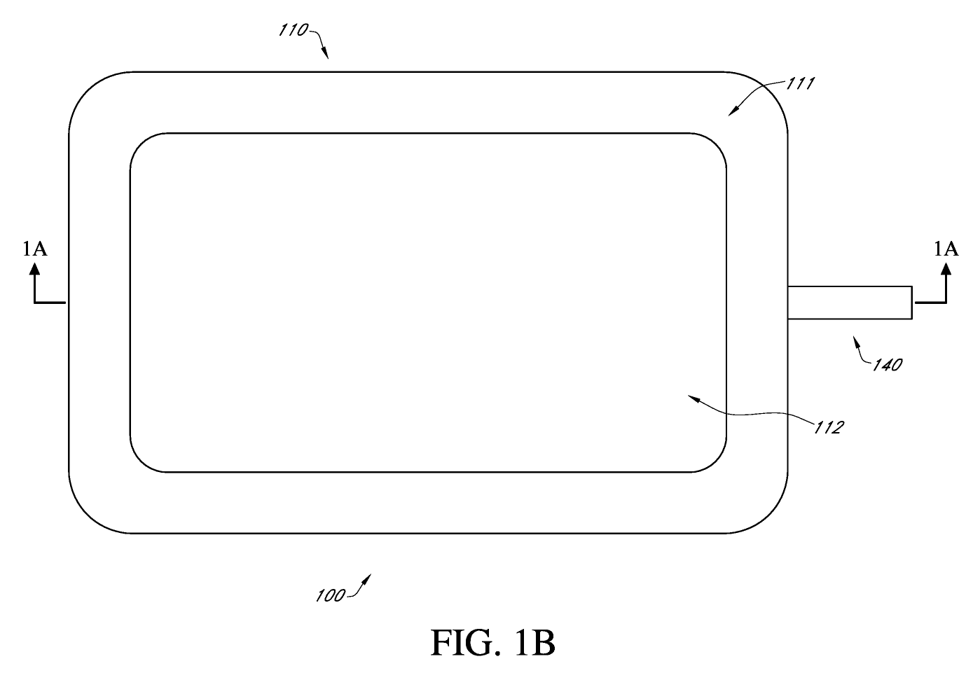

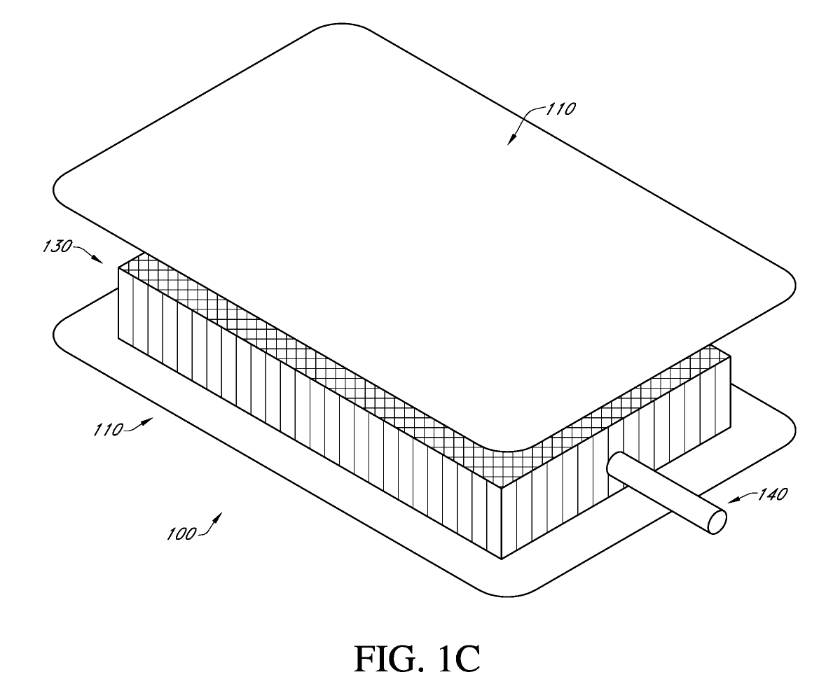

FIG. 1A illustrates a cross section of a compressible negative pressure source 100 according to an embodiment. A plan view from above the compressible negative pressure source 100 is illustrated in FIG. 1B with the line A-A indicating the location of the cross section shown in FIG. 1A. FIG. 1C illustrates an exploded view of the compressible negative pressure source 100 of FIGS. 1A-1B.

Although FIGS. 1A-C illustrate the compressible negative pressure source 100 and its component parts as having particular shapes, the construction of the layers can be applied to a compressible negative pressure source 100 that is shaped differently, for example as a square, circle, ellipse or the like. As described above, the compressible negative pressure source 100 comprises a fluid impermeable outer membrane 110 which forms an internal cavity 120, a spacer layer 130 disposed within said internal cavity 120, and which comprises one or more compressible members, and tubing 140 in communication with internal cavity 120 to provide an air leak.

The compressible negative pressure source 100 may be utilized to store and apply negative pressure to a wound, as described further below. The compressible negative pressure source 100 comprises a fluid impermeable outer membrane 110 which defines an internal cavity 120. In some embodiments the fluid impermeable outer membrane can comprise a bag defining the internal cavity. In other embodiments the fluid impermeable outer membrane can comprise an upper membrane and a lower membrane as illustrated in FIG. 1A which are sealed together along their perimeters to form an internal cavity. An upper and lower membrane can be sealed by any method known in the art, for example by an adhesive or by welding techniques.

In some embodiments the fluid impermeable outer membrane 110 may comprise one or more polymer films. In some embodiments the fluid impermeable outer membrane 110 may comprise a polyurethane film, for example Elastollan SP9109. In other embodiments the fluid impermeable outer membrane 110 may comprise any sufficiently flexible and fluid impermeable material capable of defining an internal cavity 120. As shown in FIG. 1B, the upper surface of the fluid impermeable outer membrane 110 extends outwardly away from a center of the compressible negative pressure source 100 into a border region 111 surrounding a raised central region 112 overlying the spacer layer 130, which is described further below. As indicated in FIG. 1B the general shape of the compressible negative pressure source 100 is rectangular with rounded corners. It will be appreciated that compressible negative pressure sources according to other embodiments can be shaped differently, for a compressible negative pressure source example square, circular, or elliptical, or the like.

With continued reference to FIGS. 1A-1C, in some embodiments spacer layer 130 is disposed in the internal cavity 120 formed by the fluid impermeable outer membrane 110. Spacer layer 130 comprises one or more compressible members. As described further below, the one or more compressible members may comprise a plurality of constant tension springs. In some embodiments spacer layer 130 may comprise a material having a three dimensional structure which incorporates a plurality of compressible members, for example a 3D knitted or woven fabric material which may incorporate a plurality of compressible members. In some embodiments the 3D knitted or woven fabric may be, for example Baltex 7970 weft knitted polyester.

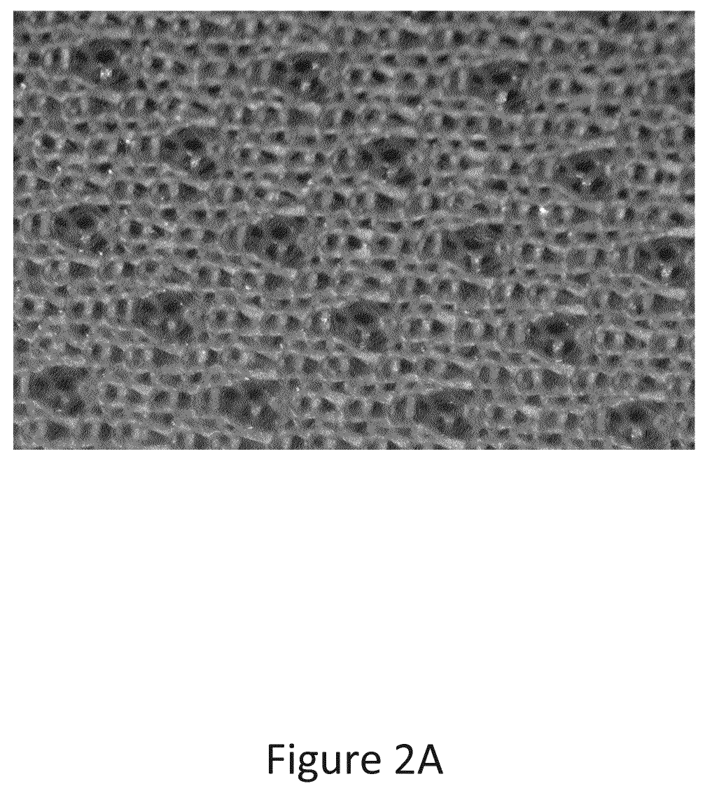

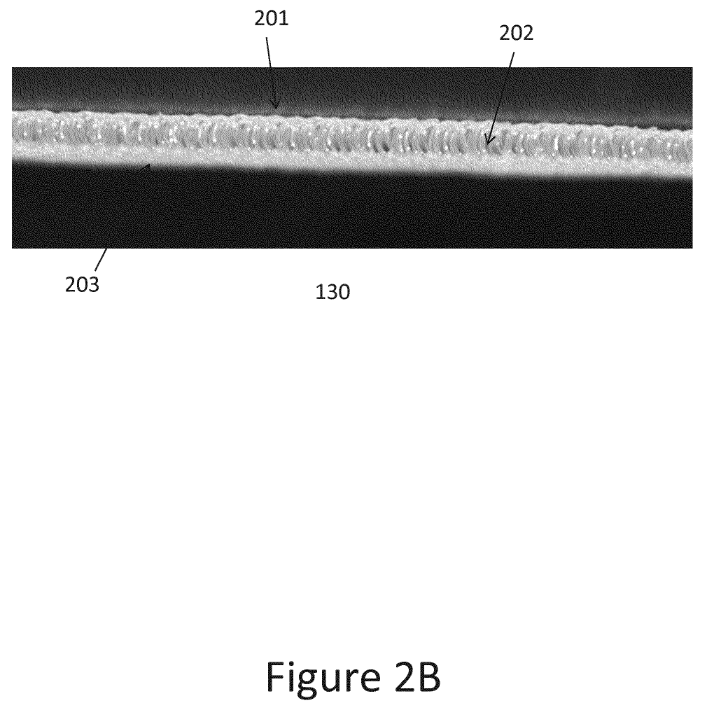

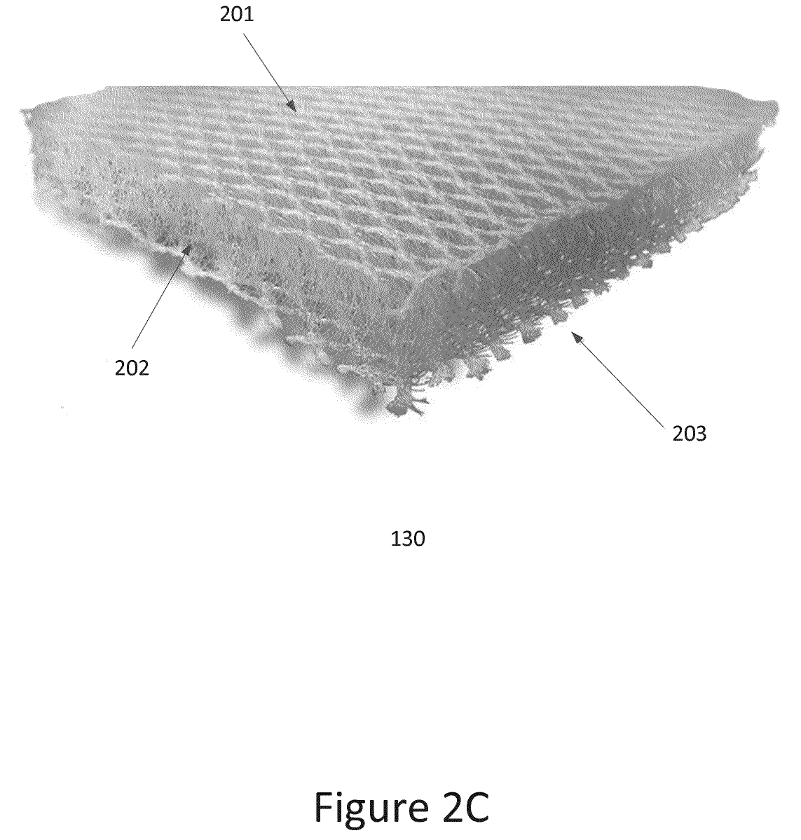

FIGS. 2A-C illustrate embodiments of spacer layer material which may be used in any of the compressible negative pressure source embodiments described herein. The spacer layer material is preferably formed of a material having a three dimensional structure, and may have a top layer (‘top layer’ is being used herein for reference only, and any such top layer may not be located above any other layers when in use) and a bottom layer (‘bottom layer’ is being used herein for reference only, and any such bottom layer may not be located below any other layers when in use) comprising a knit pattern. For example, a knitted or woven spacer fabric (for example Baltex 7970 weft knitted polyester) or a non-woven fabric could be used. In some embodiments the top and bottom layers may not comprise a knit pattern. The top and bottom layers may comprise polyester, such as 84/144 textured polyester or a flat denier polyester. The top and bottom layers may also comprise gauze, foam, felt, or other textile materials. The spacer layer material may also have a third layer formed sandwiched between the top and bottom layers, which is a region defined by a knitted polyester viscose, cellulose or the like monofilament fiber that comprises one of the one or more compressible members. In some embodiments other materials and other linear mass densities of fiber may be used in place of or in addition to the above described materials. In some embodiments, the top and bottom layers may be the same pattern and the same material, and in other embodiments they may be different patterns and/or different materials. In some embodiments the top and bottom layers may prevent one or more of the compressible members of the spacer layer from contacting or piercing the fluid impermeable membrane 110. Further, in some embodiments, the top and bottom layers may facilitate evaporation of wound exudate drawn into the dressing.

FIG. 2A illustrates a top or bottom layer of an exemplary 3D fabric spacer layer 130 in more detail. The 3D fabric spacer layer 130 comprises a top and bottom knitted layer spaced apart by the knitted structure. Rows of the knitted stitches may be referred to as a course of stitches. Columns of stitches may be referred to as a wale. A single monofilament fiber is knitted into the 3D fabric to form multiple separating strands that separate the two layers of the 3D fabric spacer layer 130. These fiber strands may act as constant tension springs and comprise one or more compressible members.

FIG. 2B illustrates a cross-section of a portion of an embodiment of spacer layer 130 that may be utilized in a compressible negative pressure source such as shown in FIGS. 1A-C. In particular, FIG. 2B illustrates a magnified view of a fabric spacer layer 130 which incorporates a plurality of compressible members. A top layer 201 of the fabric spacer layer 130 is spaced apart from the bottom layer 203. The top and bottom layers of the fabric spacer layer 130 are kept apart in a spaced apart relationship by multiple mono-filament fiber spacers 202 which act as resilient flexible pillars, or constant tension springs, separating the two layers of the fabric spacer layer 130 and comprising the one or more compressible members.

As illustrated in the side view of FIG. 2B, between the top and bottom fabric layers may be a plurality of filaments, which act as constant tension springs. The filaments may comprise a monofilament fiber or a multistrand fiber, and may be knitted polyester viscose or cellulose. The filaments, or constant tension springs, comprise the one or more compressible members of the spacer layer 130. In some embodiments, a majority of the filaments, by volume, may extend vertically (that is, perpendicular to the plane of the top and bottom layers), or substantially or generally vertically. In another embodiment, 80%-90% (or approximately 80% to approximately 90%) of the filaments or more, by volume, may extend vertically, or substantially or generally vertically. In another embodiment, all or substantially all of the filaments, by volume, may extend vertically, or substantially or generally vertically. In some embodiments, a majority, 80%-90% (or approximately 80% to approximately 90%) of the filaments or more, or even all or substantially all of the filaments, extend upward from the bottom fabric layer and/or downward from the top fabric layer, and in some embodiments, such filaments extend over a length more than half the distance between the top and bottom fabric layers. In some embodiments, a majority, 80%-90% (or approximately 80% to approximately 90%) of the filaments or more, or even all or substantially all of the filaments, span a distance that is greater in a direction perpendicular to the top and bottom fabric layers (a vertical direction) than in a direction parallel to the top and bottom fabric layers (a horizontal direction).

FIG. 2C illustrates a perspective view of an embodiment of a 3D fabric spacer layer 130. A plurality of filaments 202 which comprise the one or more compressible members may be disposed between top layer 201 and bottom layer 203 as described above with reference to FIG. 2A.

In other embodiments (not shown) the spacer layer 130 may comprise one or more compressible members which may comprise foam. For example, the one or more compressible members may comprise an open celled foam material. In some embodiments, the one or more compressible members may comprise multiple layers of open celled foam. In some embodiments, the foam is reticulated open cell foam. In some embodiments spacer layer spacer layer 130 may include two, three, four or more foam layers. The foam layers may be integrally formed, for example, by selecting a foam having a large pore size and then repeatedly dipping this to a lesser and lesser extent into material which will clog the pores or, the one or more compressible members comprising the multiple foam layers may be provided by laminating different types of foam in a layered arrangement or by securing such layers of foam in place in a known manner.

Turning again to FIG. 1A, in some embodiments the compressible negative pressure source 100 may comprise tubing 140 which provides an air leak in fluid communication with the internal cavity 120, thereby allowing air to be removed from the internal cavity 120. Due to the air impermeable nature of the fluid impermeable membrane 110, internal cavity 120 comprises a fluid and air tight cavity, with the exception of the air leak provided by tubing 140. Air which may be trapped in internal cavity 120 therefore can only exit the internal cavity 120 via the air leak provided by tubing 140.

In some embodiments tubing 140 comprises two ends; a first end which is in fluid communication with internal cavity 120 and a second end which is in fluid communication with the ambient environment. In order for air which may be contained in internal cavity 120 to be evacuated, the air must travel from internal cavity 120, into the first end of tubing 140, through the tubing 140, and out into the ambient environment through the second end of tubing 140. The generally air and fluid tight nature of fluid impermeable outer membrane 110 and thus internal cavity 120 prevents air contained in internal cavity 120 from escaping or being removed from internal cavity 120 via any other route. In embodiments where fluid impermeable outer membrane 110 comprises an upper membrane and a lower membrane the tubing 140 may be sealed between said membranes in fluid communication with the internal cavity 120. In some embodiments the tubing may pass through a hole, orifice, or aperture in the fluid impermeable outer membrane 110 to communicate with internal cavity 120. In some embodiments the tubing 140 may pass through the fluid impermeable outer membrane 110 and into the internal cavity 120. In some embodiments the tubing 140 may be in communication with the internal cavity 120 via a port or boss, as are known in the art, attached to the fluid impermeable outer membrane 110. The tubing 140 may be a single lumen conduit or may be a multi-lumen conduit.

With continued reference to FIG. 1A, the tubing 140 may additionally comprise a one-way valve (not shown) configured to prevent ambient air from flowing into internal cavity 120 via the air leak provided by tubing 140. The one-way valve may allow for the flow of air out of internal cavity 120 and into the ambient environment via tubing 140, but may prevent air from flowing into the internal cavity 120 via the tubing 140. In this way, the one-way valve serves to seal internal cavity 120 against the ambient environment so that any negative pressure within the internal cavity 120 may be maintained, as described further below, while still allowing for the removal of air from internal cavity 120 via tubing 140. The valve may additionally be configured to allow for the removal or escape of a desired amount of air from the internal cavity 120. A desired amount of air may correspond to a desired level of negative pressure in the internal cavity 120. In some embodiments the valve may comprise any such valve known in the art with the aforementioned characteristics.

In some other embodiments an air leak for internal cavity 120 may not comprise tubing 140. An air leak may comprise a hole, orifice, aperture, or port in fluid impermeable outer membrane 110 and in fluid communication with internal cavity 120. The air leak may comprise a one-way valve, as described above, incorporated directly into fluid impermeable outer membrane 110 and in fluid communication with internal cavity 120. The air leak may comprise a port, not shown, in fluid communication with the internal cavity 120 and configured to communicate with a source of negative pressure as described herein. The port may be attached to the fluid impermeable outer membrane 110. The port may be attached to the fluid impermeable outer membrane 110 using techniques known in the art, such as adhesive or welding. In some embodiments the port comprises a soft polymer, for example a polyethylene, a polyvinyl chloride, a silicone or polyurethane having a hardness of 30 to 90 on the Shore A scale. In some embodiments the port is situated over or in a hole, aperture, or orifice in the fluid impermeable outer membrane 110, to thereby fluidically communicate with the internal cavity 120.

In operation, the evacuation of air which may be contained within the internal cavity 120 is effectuated in a variety of ways. As described above, air is only able to escape the internal cavity 120 via the air leak, which in the exemplary embodiment illustrated in FIGS. 1A-C is provided by tubing 140. Air may be forced out of internal cavity 120 through tubing 140 by physically compressing the compressible negative pressure source 100 which in turn physically compresses internal cavity 120. The physical compression of internal cavity 120 effectively reduces the volume of internal cavity 120, thereby forcing any air which may have been contained in internal cavity 120 out through the air leak, here tubing 140. Physical compression of internal cavity 120 can be achieved by, for example squeezing compressible negative pressure source 100. A user may, for example, hold the compressible negative pressure source 100 in a hand and squeeze the compressible negative pressure source 100 in order to evacuate air from internal cavity 120. Alternatively, a user may, for example, exert pressure on a surface, for example the top surface, of compressible negative pressure source 100 while the compressible negative pressure source 100 is positioned against a relatively stationary and resistant surface, for example the body of a user. In some embodiments a valve, as described above, may prevent ambient air from flowing back into internal cavity 120 via the air leak once air has been removed from internal cavity 120.

A negative pressure source (not shown) may be used to remove air from internal cavity 120 via tubing 140. The negative pressure source, for example a syringe, may be connected to the end of tubing 140 in communication with the ambient environment in order to establish a fluidic connection between the negative pressure source and internal cavity 120. The negative pressure source may then apply negative pressure to the internal cavity 120 via the tubing 140. For example, an end of a syringe may be connected to tubing 140 and the plunger of the syringe may be drawn out in order to apply negative pressure to the internal cavity 120. The air which may have been previously contained within internal cavity 120 is drawn out of internal cavity 120 and through tubing 140 by the negative pressure source. In some embodiments a valve, as described above, may prevent ambient air from flowing back into internal cavity 120 via the air leak. In some other embodiments where compressible negative pressure source 100 comprises a port, valve, or hole in fluid impermeable outer membrane 110, a negative pressure source may be attached to said port, valve, or hole to thereby establish fluid communication between said negative pressure source and internal cavity 120. In these other embodiments, upon removal of a desired amount of air from the internal cavity 120 the negative pressure source may optionally be removed from fluid communication with the internal cavity 120. The source of negative pressure may comprise a vacuum pump, of any kind known in the art. The pump may be manually operated, while in others the pump may be automated or electrically driven. In some embodiments the source of negative pressure may comprise a piston pump. In other embodiments the source of negative pressure may comprise a manually operated syringe.

Utilizing any suitable mechanism, the removal of a sufficient amount of air from the internal cavity 120 causes spacer layer 130 comprising one or more compressible members to compress. Thus, in some embodiments the spacer layer 130 as shown in FIGS. 1A-C is configured to compress upon the removal of air by the application of a negative pressure to the internal cavity 120. In some embodiments the spacer layer 130 is configured to compress upon the application of a minimum level of negative pressure to the internal cavity 120. This minimum level of negative pressure may at least equal to the level of negative pressure applied by an expansion force of the one or more compressible members, as described further below. In some embodiments the minimum level of negative pressure is greater than the level of negative pressure applied by the expansion force of the one or more compressible members. In some embodiments the one or more compressible members are configured not to compress until a negative pressure of at least −45 mmHg is applied to the internal cavity 120. In other embodiments the one or more compressible members are configured not to compress until a negative pressure of at least −70 mmHg is applied to the internal cavity 120. In even further embodiments the one or more compressible members are configured not to compress until a negative pressure of at least −400 mmHg is applied to the internal cavity 120.

The compressed one or more compressible members which comprise the spacer layer 130 are thereafter configured to exert an expansion force within the internal cavity 120 that generates a level of negative pressure which may be communicated to a desired location, for example a wound. In some embodiments the compression of one or more compressible members results in elastic deformation of the one or more compressible members, and therefore the expansion force exerted by the one or more compressible members corresponds to the stiffness of the one or more compressible members. Preferably, the stiffness of the one or more compressible members is such that upon compression, the compressed members are sufficiently stiff to exert an expansion force that can be used to apply a desired level of negative pressure to a desired location. However, the one or more compressible members are preferably not so stiff that the removal of air from the internal cavity 120 is unable to compress the one or more compressible members. As the expansion force exerted by the one or more compressible members corresponds to the stiffness of the one or more compressible members, the expansion force exerted by the one or more compressible members can be varied by selecting one or more compressible members with an appropriate stiffness.

In some embodiments the expansion force generated by the one or more compressible members against the internal cavity 120 can be configured to apply a negative pressure of at least −45 mmHg to a desired location. In other embodiments the expansion force generated by the one or more compressible members against the internal cavity 120 is configured to apply a negative pressure of at least −70 mmHg to a desired location. In even further embodiments the expansion force generated by the one or more compressible members against the internal cavity 120 is configured to apply a negative pressure of between about −20 mmHg to about −200 mmHg to a desired location. In some embodiments the expansion force generated by the one or more compressible members against the internal cavity 120 is configured to apply a negative pressure of up to −400 mmHg to a desired location.

The expansion force generated by the one or compressible members against the internal cavity can be used to communicate negative pressure to a desired location such as a wound through any suitable outlet. One possible outlet may be a tubing connected to the compressible negative pressure source 100. For example, the tubing 140 shown in FIGS. 1A-1C, which was previously described as providing an air leak, could alternatively provide the conduit to communicate negative pressure to a wound. In such an embodiment, the tubing 140 may include a valve as described above to regulate the amount of negative pressure that is communicated through the tubing to the wound. In some embodiments, separate tubings or conduits may be provided for the air leak and to communicate negative pressure, each of which may include their own valves.

In some embodiments the compressible negative pressure source 100 further comprises an audible alarm system configured to sound when the pressure in the internal cavity 120 rises above a predetermined negative pressure. In some embodiments the audible alarm system is located in the tubing 140 of the compressible negative pressure source 100. In other embodiments the audible alarm system is not located in the tubing. In some embodiments the audible alarm system comprises a valve which emits a sound at a predetermined negative pressure, to thereby indicate that the pressure in the internal cavity 120 has risen above a predetermined level. In some embodiments the audible alarm system does not comprise electronic components. In some embodiments the audible alarm may comprise electronic components, for example a speaker.

In some embodiments the compressible negative pressure source 100 may further comprise a light emitting alarm system, configured to emit light when the pressure in the internal cavity 120 rises above a predetermined negative pressure. In other embodiments the compressible negative pressure source 100 may comprise a light emitting alarm and may not comprise an audible alarm. In certain embodiments the audible alarm system and the light emitting alarm system are integral with each other. The light emitting alarm may include a light source, for example, a light emitting diode (LED). In some embodiments the light emitting alarm may comprise a sensor to detect when the pressure in the internal cavity 120 rises above a predetermined negative pressure. The light source may be located on an exterior surface of the compressible negative pressure source 100, so that it is visible to a user when emitting light. For example, the light source may be located on the fluid impermeable outer membrane 110.

In some embodiments the compressible negative pressure source 100 is configured to be wearable, for example on the body of a user. In some embodiments the compressible negative pressure source 100 is configured to be secured to the body of a user. In some embodiments the compressible negative pressure source 100 further comprises an adhesive, not shown, on the fluid impermeable outer membrane 110 which is configured to adhere the compressible negative pressure source 100 to the body of a user when the compressible negative pressure source 100 is in use. In some embodiments the adhesive on the fluid impermeable outer membrane 110 may be configured to adhere the compressible negative pressure source 100 to the skin of a user.

In some embodiments the compressible negative pressure source 100 may further comprise a pressure monitor, not shown, such as the pressure monitor described in U.S. Pat. No. 7,846,141, which is herein incorporated by reference in its entirety. In some embodiments the pressure monitor may comprise a plurality of protrusions on the fluid impermeable outer membrane 110 for monitoring pressure in the internal cavity 120. In some embodiments the plurality of protrusions are spaced apart and are configured to indicate whether a predetermined level of negative pressure has been achieved in the internal cavity 120.

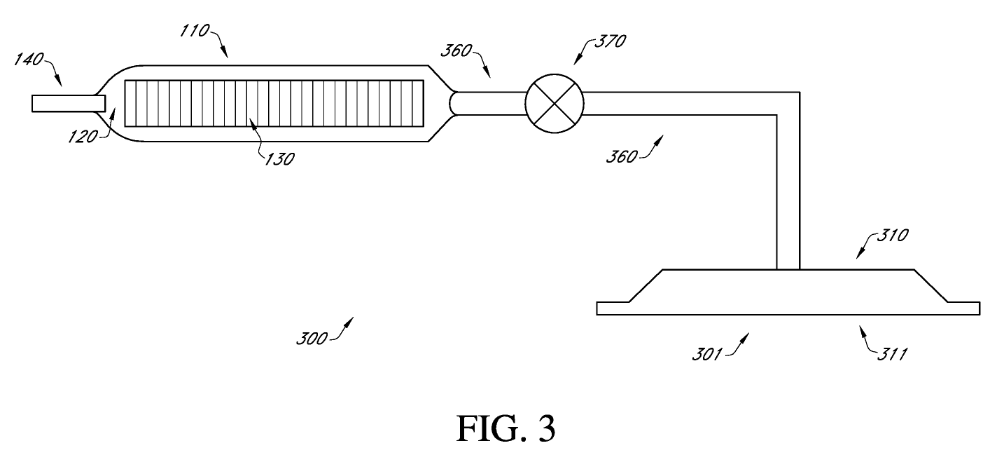

FIG. 3 illustrates an embodiment of a wound treatment apparatus 300 including a compressible negative pressure source 100 similar to the compressible negative pressure source illustrated in FIGS. 1A-C, and a wound dressing 310. The wound dressing 310 may be, for example a wound dressing as described in U.S. Pat. No. 9,061,095; which is hereby incorporated by reference in its entirety. The wound dressing 310 may also be, for example a wound dressing as described in International Application PCT/IB2013/002060 and published as WO 20140/20440 A1 which is hereby incorporated by reference in its entirety. Wound dressing 310 may also comprise one or more adhesive, liquid impermeable backing layers configured to maintain a seal over the wound.

In some embodiments the compressible negative pressure source 100 comprises a conduit 360 in fluid communication with the internal cavity 120 of the compressible negative pressure source 100. The conduit 360 communicates negative pressure generated by the expansion force of the one or more compressible members of compressible negative pressure source 100 to a desired location, for example a wound dressing 310. In embodiments where the fluid impermeable outer membrane 110 comprises an upper membrane and a lower membrane the conduit 360 may be sealed between said membranes in fluid communication with the internal cavity 120. In some embodiments the conduit 360 may pass through a hole, orifice, or aperture in the fluid impermeable outer membrane 110, and may or may not extend into the internal cavity 120. In some embodiments the conduit 360 may be in communication with the internal cavity 120 via a port or boss, as are known in the art, attached to the fluid impermeable outer membrane 110. The conduit 360 may be a single lumen conduit or may be a multi-lumen conduit. The compressible negative pressure source 100 is therefore in fluid communication with the wound dressing 310 via the conduit 360. In some embodiments the wound dressing 310 is configured to be positioned over a wound and the compressible negative pressure source 100 is configured to apply negative pressure to the wound through the separate wound dressing 310. Both the wound dressing 310 and the compressible negative pressure source 100 may be configured to be wearable on the body of a user, as described herein above, while wound treatment apparatus 310 is in use.

As illustrated in FIG. 3, the wound treatment apparatus 300 may further comprise a regulator valve 370 disposed between wound dressing 310 and the compressible negative pressure source 100, and in fluid communication with both wound dressing 310 and the internal cavity 120 of compressible negative pressure source 100. The conduit 360 which connects the wound dressing 310 to the compressible negative pressure source 100 may further comprise the regulator valve 370. The regulator valve 370 may be configured to regulate the negative pressure supplied to the wound dressing 310 by the internal cavity 120. That is, in some embodiments regulator valve 370 is configured to selectively open and close so as to regulate the level of negative pressure supplied to the wound dressing 310 from the compressible negative pressure source 100. In some embodiments regulator valve 370 is substantially the same as, and functions substantially the same as the reservoir valve described in U.S. Pat. No. 9,180,231, which is hereby incorporated by reference in its entirety.

In some embodiments, where wound treatment apparatus 300 comprises a regulator valve 370, compressible negative pressure source 100 may act as a negative pressure reservoir. For example, compressible negative pressure source 100 may be configured to apply a negative pressure to the regulator valve 370 that is higher than may be desirable to be applied to the wound dressing 310. The regulator valve 370 is configured to regulate the level of negative pressure applied to the wound dressing so that a desired level of negative pressure is applied to the wound dressing 310. The regulator valve 370 may be configured to selectively open and close in response to the negative pressure of the wound dressing 310, such that when the negative pressure of the wound dressing 310 rises above a predetermined threshold the regulator valve 370 is configured to open and communicate negative pressure from the compressible negative pressure source 100 to the wound dressing 310. Once the negative pressure of the wound dressing 310 has reached a predetermined level the regulator valve 370 is configured to close and to stop, or sufficiently regulate negative pressure from the compressible negative pressure source 100 so as to prevent an undesirably large amount of negative pressure from being applied to the wound dressing 310.

The apparatus 300 as illustrated in FIG. 3 may be used to treat a wound site on a patient. The healthy skin surrounding the wound site 301 is preferably cleaned and excess hair removed or shaved. The wound site 301 may also be irrigated with sterile saline solution if necessary. Optionally, a skin protectant may be applied to the skin surrounding the wound site 301. If necessary, a wound packing material, such as foam or gauze, may be placed in the wound site 301. This may be preferable if the wound site 301 is a deeper wound.

After the skin surrounding the wound site 301 is dry the wound dressing 310 may be positioned and placed over the wound site 301. Preferably, the wound dressing 310 is placed with the wound contact layer 311 (if present) over and/or in contact with the wound site 301. In some embodiments, an adhesive layer is provided on the lower surface of the wound contact layer 311, which may in some cases be protected by an optional release layer to be removed prior to placement of the wound dressing 310 over the wound site 301. Conduit 360 may be fluidly connected to the dressing using any suitable method. In one embodiment, the dressing 310 is positioned such that the connection between the conduit 360 and the dressing 310 is in a raised position with respect to the remainder of the dressing 310 so as to avoid fluid pooling around the connection. In some embodiments, the dressing 310 is positioned so that the connection between the conduit 360 and the dressing 310 is not directly overlying the wound, and is level with or at a higher point than the wound. In one embodiment, a soft flexible port such as described in U.S. Pat. No. 9,226,737 and PCT Patent Publication No. WO 2013/175306 A1, which are hereby incorporated by reference in their entireties, may be utilized to connect the conduit 360 to the dressing. To help ensure adequate sealing for TNP, the edges of the dressing 310 are preferably smoothed over to avoid creases or folds.

With continued reference to FIG. 3, the compressible negative pressure source 100 may then be connected to the wound dressing 310 via conduit 360, or the conduit 360 may have already been connected to the compressible negative pressure source 100. A first end of conduit 360 may be connected to the wound dressing 310 to provide fluid communication between the wound site 301 and the conduit 360 through the wound dressing 310. A second end of the conduit 360 may be connected to the compressible negative pressure source 100 such that it is in fluid communication with the internal cavity 120. The wound dressing 310 may be connected to conduit 360 prior to compressible negative pressure source 100, or compressible negative pressure source 100 may be connected to the conduit 360 prior to wound dressing 310, or both the wound dressing 310 and compressible negative pressure source 100 may be connected to conduit 360 simultaneously. Alternatively, in some embodiments the wound treatment apparatus 300 may be provided as a single integral unit configured to be applied to a wound site with the wound dressing 310 and compressible negative pressure source 100 already connected via conduit 360.

After wound dressing 310 has been secured over the wound site 301 on the body of a user, the compressible negative pressure source 100 may then optionally be secured to the body of the user at a location substantially near to the wound site 301. Preferably the compressible negative pressure source 100 is secured to body of the user at a location that is near enough to the wound site 301 and wound dressing 310 so that conduit 360 is able to span the distance between the compressible negative pressure source 100 and the wound dressing 310. The location of the compressible negative pressure source 100 on the body of the user is preferably selected to facilitate ease of use of the compressible negative pressure source 100 with respect to compression of the compressible negative pressure source 100, whether by squeezing, physical pressure, or a negative pressure source. As described above, the compressible negative pressure source 100 may comprise an adhesive on the lower surface of fluid impermeable outer membrane 110 to adhere the compressible negative pressure source 100 to the skin of a user. Any alternative means of securing compressible negative pressure source 100 to the skin or body of a user as are known in the art may also be used. Additionally, the length of conduit 360 may be selected based on a preferable location of the compressible negative pressure source 100 relative to the location of wound dressing 310 on the user so as to prevent kinking due to excess length of conduit 360.

Once the wound dressing 310 has been secured over wound site 301, compressible negative pressure source 100 has optionally been secured to the body of the user, and wound dressing 310 has been connected to compressible negative pressure source 100 via conduit 360, the compressible negative pressure source 100 can be operated to supply negative pressure to the wound dressing 310. A sufficient amount of air is removed from the internal cavity 120 of compressible negative pressure source 100 by any suitable mechanism as described above. For example, a user may apply even pressure to the surface of the compressible negative pressure source 100 with a hand, or a negative pressure source such as a syringe may be connected to the compressible negative pressure source 100. The removal of a sufficient amount of air causes the spacer layer 130 comprising one or more compressible members to compress as described above. The compressed one or more compressible members which comprise the spacer layer 130 are thereafter configured to exert an expansion force within the internal cavity 120 that generates a level of negative pressure which is then communicated to the wound dressing 310 via the conduit. The negative pressure is then applied to the wound site 301 by the wound dressing 310.

Treatment of the wound site 301 preferably continues until the wound has reached a desired level of healing. The level of negative pressure at the wound site 301 may diminish over time, which may be due in part to small leaks which may occur in the seal between the wound dressing 310 and the skin surrounding the wound site 301. As the level of negative pressure at the wound site 301 diminishes, the compressible negative pressure source 100 may again be compressed in order to continue to apply a desired level of negative pressure to the wound site 301 via the wound dressing. The compressible negative pressure source 100 can be operated as described above multiple times to continue to apply negative pressure to the wound site 301, as may be deemed necessary by one of skill in the art. In some embodiments, it may be desirable to replace the wound dressing 310 after a certain time period has elapsed, or if the dressing is full of wound fluids. During such changes, the compressible negative pressure source 100 may be kept, with just the wound dressing 310 being changed.

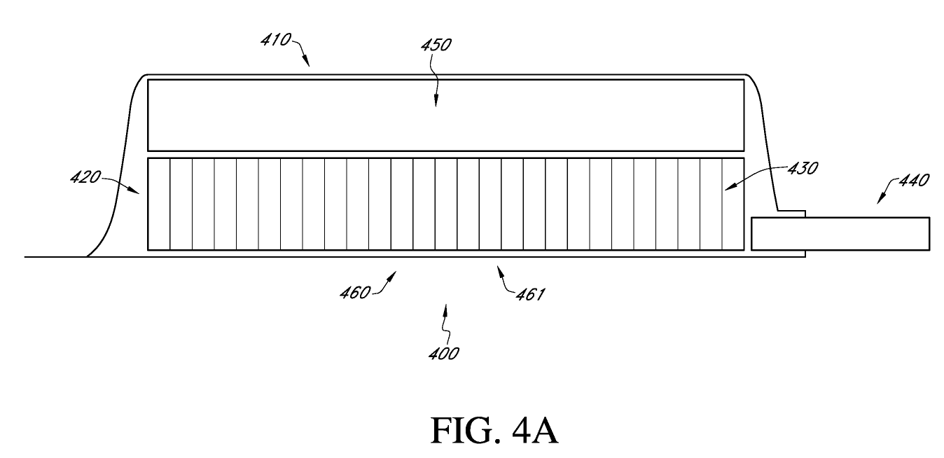

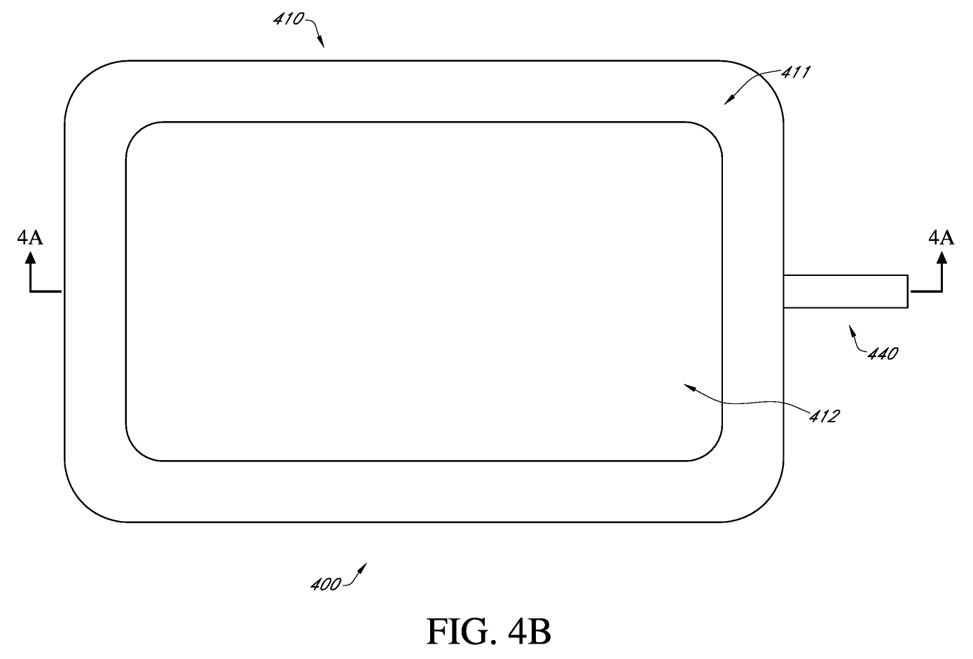

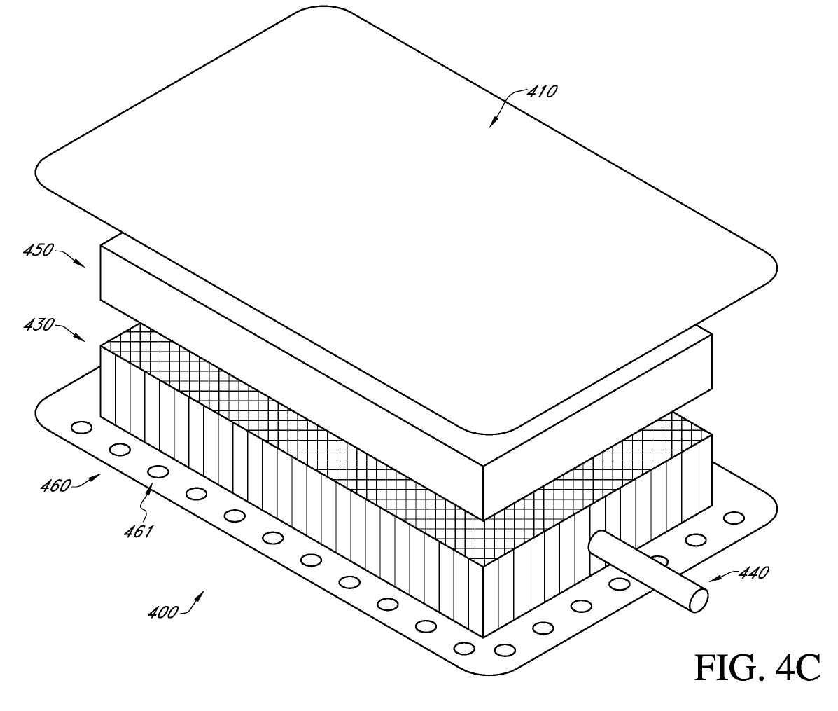

FIG. 4A illustrates a cross section through a wound dressing 400 according to another embodiment. A plan view from above the wound dressing 400 is illustrated in FIG. 4B with the line B-B indicating the location of the cross section shown in FIG. BA. FIG. 4C illustrates an exploded view of the wound dressing 400 of FIGS. 4A and 4B.

Referring to FIG. 4A, the wound dressing 400 comprises an optional wound contact layer 460 and a fluid impermeable outer membrane 410. The wound contact layer 460 and the fluid impermeable outer membrane 410 may be adhered to each other and define an internal cavity 420. A transmission layer 430 comprising one or more compressible members is disposed in the internal cavity 420 of the wound dressing 400, similar to those described above and further described below. In some embodiments the wound dressing 400 further comprises an absorbent layer 450 disposed in the internal cavity 420 and over the transmission layer 430. In some other embodiments the absorbent layer 450 may be disposed under the transmission layer 430. In some embodiments, the absorbent layer 450 may be disposed between one or more upper transmission layers and one more lower transmission layers. The wound dressing 400 can be located over a wound site to be treated. The dressing 400 forms a sealed cavity over the wound site. An adhesive may be provided on a lower surface of the outer membrane and/or the wound contact layer 460 to adhere the wound dressing to skin surrounding a wound.

In some embodiments, it may be preferable for the wound site to be filled partially or completely with a wound packing material. This wound packing material is optional, but may be desirable in certain wounds, for example deeper wounds. The wound packing material can be used in addition to the wound dressing 400. The wound packing material generally may comprise a porous and conformable material, for example foam (including reticulated foams), or gauze. Preferably, the wound packing material is sized or shaped to fit within the wound site so as to fill any empty spaces. The wound dressing 400 may then be placed over the wound site and wound packing material overlying the wound site. When a wound packing material is used, once the wound dressing 400 is sealed over the wound site, negative pressure is transmitted from the wound dressing 400, through the wound packing material, and to the wound site. This negative pressure draws wound exudate and other fluids or secretions away from the wound site.

With continued reference to FIG. 4A, in embodiments comprising a wound contact layer 460, the wound contact layer 460 can be a polyurethane layer or polyethylene layer or other flexible layer which is perforated, for example via a hot pin process, laser ablation process, ultrasound process or in some other way or otherwise made permeable to liquid and gas. The perforations 461 are through holes in the wound contact layer 460 which enables fluid to flow through the layer. The wound contact layer 460 helps prevent tissue ingrowth into the other material of the wound dressing. The perforations 461 are small enough to meet this requirement but still allow fluid through. For example, perforations formed as slits or holes having a size ranging from 0.025 mm to 1.2 mm are considered small enough to help prevent tissue ingrowth into the wound dressing while allowing wound exudate to flow into the dressing. The wound contact layer 461 helps hold the whole wound dressing together and helps to create an air tight seal around the absorbent pad in order to maintain negative pressure at the wound. The wound contact layer 461 also acts as a carrier for an optional adhesive layer (not shown). For example, a pressure sensitive adhesive may be provided on the underside surface of the wound contact layer 460. The pressure sensitive adhesive, which may be a silicone, hot melt, hydrocolloid or acrylic based adhesive or other such adhesives. When an adhesive is utilized this helps adhere the wound dressing 400 to the skin around a wound site.

In some embodiments the fluid impermeable outer membrane 410 is substantially the same as the fluid impermeable outer membrane 110 described herein above. In some embodiments the fluid impermeable outer membrane 410 is sealed to the wound contact layer 460 along their perimeters to form an internal cavity 420. As can be seen in FIG. 4B, the upper surface of the fluid impermeable outer membrane 410 extends outwardly away from a center of the dressing into a border region 411 surrounding a central raised region 412 overlying the transmission layer 430 and the absorbent layer 450. As indicated in FIG. 4B, the general shape of the wound dressing 400 is rectangular with rounded corner regions. It will be appreciated that wound dressings according to other embodiments of the present invention can be shaped differently such as square, circular or elliptical dressings, or the like.

As illustrated in FIG. 4A, the transmission layer 430 comprising one or more compressible members may be substantially the same as spacer layer 130 described herein above with respect to FIGS. 1A-C. Transmission layer 430 may comprise one or more compressible members. As described further below, the one or more compressible members may comprise a plurality of constant tension springs. In some embodiments transmission layer 430 may comprise a material having a three dimensional structure which incorporates a plurality of compressible members, for example a 3D knitted or woven fabric material which may incorporate a plurality of compressible members. In some embodiments the 3D knitted or woven fabric may be, for example Baltex 7970 weft knitted polyester.

In some embodiments, transmission layer 430 may comprise a 3D polyester transmission layer including a top layer (‘top layer’ is being used herein for reference only, and any such top layer may not be located above any other layers when in use) which is a 84/144 textured polyester, and a bottom layer (‘bottom layer’ is being used herein for reference only, and any such bottom layer may not be located below any other layers when in use) which is a 100 denier flat polyester and a third layer formed sandwiched between these two layers which is a region defined by a knitted polyester viscose, cellulose or the like monofilament fiber that comprises one of the one or more compressible members. In some embodiments other materials and other linear mass densities of fiber may be used in place of or in addition to the above described materials.

A 3D fabric transmission layer 430 may incorporate a plurality of compressible members. A top layer of the fabric transmission layer 430 is spaced apart from the bottom layer. The top and bottom layers of the fabric transmission layer 430 are kept apart in a spaced apart relationship by multiple mono-filament fiber spacers which act as resilient flexible pillars, or constant tension springs, separating the two layers of the fabric transmission layer 430 and comprising the one or more compressible members.

A plurality of filaments, which may act as constant tension springs may be disposed between the top and bottom fabric layers of transmission layer 430. The filaments may comprise a monofilament fiber or a multistrand fiber, and may be knitted polyester viscose or cellulose. The filaments, or constant tension springs, comprise the one or more compressible members of the transmission layer 430. In some embodiments, a majority of the filaments, by volume, may extend vertically (that is, perpendicular to the plane of the top and bottom layers), or substantially or generally vertically. In another embodiment, 80%-90% (or approximately 80% to approximately 90%) of the filaments or more, by volume, may extend vertically, or substantially or generally vertically. In another embodiment, all or substantially all of the filaments, by volume, may extend vertically, or substantially or generally vertically. In some embodiments, a majority, 80%-90% (or approximately 80% to approximately 90%) of the filaments or more, or even all or substantially all of the filaments, extend upward from the bottom fabric layer and/or downward from the top fabric layer, and in some embodiments, such filaments extend over a length more than half the distance between the top and bottom fabric layers. In some embodiments, a majority, 80%-90% (or approximately 80% to approximately 90%) of the filaments or more, or even all or substantially all of the filaments, span a distance that is greater in a direction perpendicular to the top and bottom fabric layers (a vertical direction) than in a direction parallel to the top and bottom fabric layers (a horizontal direction).

The transmission layer 430, as illustrated in FIG. 4A, is located above the wound contact layer 460. This layer, in addition to comprising one or more compressible members, is further configured to allow transmission of fluid including liquid and gas away from a wound site into the above layer or layers of the wound dressing 400. In particular, the transmission layer 430 ensures that an open air channel can be maintained to communicate negative pressure over the wound area even when the absorbent layer has absorbed substantial amounts of exudates. The layer should remain open under the typical pressures that will be applied during negative pressure wound therapy as described above, so that the whole wound site sees an equalized negative pressure.

In some embodiments the top fabric layer of transmission layer 430 may have more filaments in a yarn used to form it than the number of filaments making up the yarn used to form the bottom fabric layer, in order to control moisture flow across the transmission layer 430. Particularly, by having a filament count greater in the top layer, that is to say, the top layer is made from a yarn having more filaments than the yarn used in the bottom layer, liquid tends to be wicked along the top layer more than the bottom layer. The orientation of such filaments may promote vertical wicking of fluid through the transmission layer 430. In some embodiments, the ratio of the amount of fluid wicked vertically through the transmission layer material to the amount of fluid wicked laterally across the transmission layer material when under negative pressure may be 2:1 or more, or approximately 2:1 or more, or may be up to 10:1 or more, or approximately 10:1 or more, in some embodiments.

Preferably, to improve the liquid flow across the transmission layer 430 (that is to say perpendicular to the channel region formed between the top and bottom spacer layers, the 3D fabric is treated with a dry cleaning agent (such as, but not limited to, Perchloro Ethylene) to help remove any manufacturing products such as mineral oils, fats and/or waxes used previously which might interfere with the hydrophilic capabilities of the transmission layer. In some embodiments, an additional manufacturing step can subsequently be carried in which the 3D transmission layer fabric is washed in a hydrophilic agent (such as, but not limited to, Feran Ice 30 g/l available from the Rudolph Group). This process step helps ensure that the surface tension on the materials is so low that liquid such as water can enter the fabric as soon as it contacts the 3D knit fabric. This also aids in controlling the flow of the liquid insult component of any exudates.

In some embodiments the wound dressing 400 comprises an absorbent layer 450 disposed over the transmission layer 430 and inside the internal cavity 420. In some other embodiments the absorbent layer 450 may be disposed under the transmission layer 430, or may be positioned between multiple transmission layers. In some embodiments the absorbent layer 450 is substantially identical to absorbent layer 110 as shown in, for example FIGS. 1A and 1B of U.S. Pat. No. 9,061,095, which is hereby incorporated by reference in its entirety. In other embodiments the absorbent layer 450 may be substantially identical to absorbent layer 2110 as shown in, for example FIGS. 3A-B; absorbent layer 402 as shown in, for example FIGS. 5-12; absorbent layer 2308 as shown in, for example FIGS. 24A-F; absorbent layer 503 as shown in, for example FIGS. 25A-B; absorbent layer 3440 as shown in, for example FIG. 34A-B; and absorbent layer 3940 as shown in, for example FIG. 39A-B of International Application PCT/IB2013/002060, published as WO 20140/20440 A1 which is hereby incorporated by reference in its entirety.

With reference again to FIG. 4A, the absorbent layer 450 is configured to collect wound exudate from the wound that passes through the wound contact layer 460 and through the transmission layer 430. The absorbent layer 450 may comprise a foam or non-woven natural or synthetic material and may optionally include or be super-absorbent material. In use the absorbent layer 450 forms a reservoir for fluid, particularly liquid, removed from the wound site and draws those fluids towards the fluid impermeable outer membrane 410. The material of the absorbent layer also prevents liquid collected in the wound dressing from flowing in a sloshing manner. The absorbent layer 450 also helps distribute fluid throughout the layer via a wicking action so that fluid is drawn from the wound site and stored throughout the absorbent layer. This helps prevent agglomeration in areas of the absorbent layer. The capacity of the absorbent material must be sufficient to manage the exudates flow rate of a wound when negative pressure is applied. Since in use the absorbent layer experiences negative pressures the material of the absorbent layer is chosen to absorb liquid under such circumstances. A number of materials exist that are able to absorb liquid when under negative pressure, for example superabsorber material. The absorbent layer 110 may typically be manufactured from ALLEVYN™ foam, Freudenberg 114-224-4 and/or Chem-Posite™11C-450.

Turning again to FIG. 4A, in some embodiments the wound dressing 400 may comprise tubing 440 which provides an air leak in fluid communication with the internal cavity 420, thereby allowing air to be removed from the internal cavity 420. Due to the air impermeable nature of the fluid impermeable membrane 410, internal cavity 420 comprises a fluid and air tight cavity, with the exception of the air leak provided by tubing 440. Air which may be trapped in internal cavity 420 therefore can only exit the internal cavity 420 via the air leak provided by tubing 440. The tubing 440 may be substantially similar to, and operate in substantially the same manner as the tubing 140 described with reference to FIGS. 1A-C above.

The tubing 440 comprises two ends; a first end which is in fluid communication with internal cavity 420 and a second end which is in fluid communication with the ambient environment. In order for air which may be contained in internal cavity 420 to be evacuated, the air must travel from internal cavity 420, into the first end of tubing 440, through the tubing 440, and out into the ambient environment through the second end of tubing 440. With continued reference to FIG. 4A, the tubing 440 may additionally comprise a one-way valve (not shown) configured to prevent ambient air from flowing into internal cavity 420 via the air leak provided by tubing 440. The one-way valve may be substantially the same as, and operate in substantially the same manner as the one-way valve as described above with reference to FIGS. 1A-C. The one-way valve may allow for the flow of air out of internal cavity 420 and into the ambient environment via tubing 440, but may prevent air from flowing into the internal cavity 420 via the tubing 440.

In some other embodiments an air leak for internal cavity 420 may not comprise tubing 440. An air leak may comprise a hole, orifice, or aperture in fluid impermeable outer membrane 410 and in fluid communication with internal cavity 420. Such an air leak may be substantially identical to the air leak as described above with reference to FIGS. 1A-C.

Wither reference now to FIG. 4C, as illustrated the fluid impermeable outer membrane 410 in this embodiment does not comprise a port for connecting the wound dressing 400 to an external source of negative pressure. Unlike existing wound dressings, a port for connection with an external source of negative pressure is not needed in wound dressing 400 because negative pressure is not applied to the wound from an external source. Instead, as described further below, negative pressure is applied to the wound directly by the dressing itself.

Turning again to FIG. 4A, in operation, the evacuation of air which may be contained within the internal cavity 420 can effectuated in a variety of ways. As described above, air is only able to escape the internal cavity 420 via the air leak, which in the exemplary embodiment illustrated in FIGS. 4A-C is provided by tubing 440. Air may be forced out of internal cavity 420 through tubing 440 by physically compressing the wound dressing 400 which in turn physically compresses internal cavity 420. The physical compression of internal cavity 420 effectively reduces the volume of internal cavity 420, thereby forcing any air which may have been contained in internal cavity 420 out through the air leak, here tubing 440. Physical compression of internal cavity 420 can be achieved by mechanisms similar to those described above with reference to the compressible negative pressure source 100 of FIGS. 1A-C, for example squeezing wound dressing 400. A user may, for example, exert pressure on a surface, for example the top surface, of wound dressing 400 while the dressing is positioned over a wound site. In some embodiments a valve, as described above, may prevent ambient air from flowing back into internal cavity 420 via the air leak once air has been removed from internal cavity 420.

In some other embodiments a negative pressure source (not shown) may alternatively optionally be used to remove air from internal cavity 420 via tubing 440. The negative pressure source, for example a syringe, may be connected to the end of tubing 440 in communication with the ambient environment in order to establish a fluidic connection between the negative pressure source and internal cavity 420. The negative pressure source may then apply negative pressure to the internal cavity 420 via the tubing 440. For example, an end of a syringe may be connected to tubing 440 and the plunger of the syringe may be drawn out in order to apply negative pressure to the internal cavity 420. The air which may have been previously contained within internal cavity 420 is drawn out of internal cavity 420 and through tubing 440 by the negative pressure source. In some embodiments a valve, as described above, may prevent ambient air from flowing back into internal cavity 420 via the air leak. In these other embodiments, upon removal of a desired amount of air from the internal cavity 420 the negative pressure source may optionally be removed from fluid communication with the internal cavity 420. The source of negative pressure may comprise a vacuum pump, of any kind known in the art. The pump may be manually operated, while in others the pump may be automated or electrically driven. In some embodiments the source of negative pressure may comprise a piston pump. In other embodiments the source of negative pressure may comprise a manually operated syringe.

With continued reference to FIG. 4A, utilizing any suitable mechanism, the removal of a sufficient amount of air from the internal cavity 420 causes transmission layer 430 comprising one or more compressible members to compress. Thus, in some embodiments the transmission layer 430 as shown in FIGS. 4A-C is configured to compress upon the removal of air by the application of a negative pressure to the internal cavity 420. In some embodiments the transmission layer 430 is configured to compress upon the application of a minimum level of negative pressure to the internal cavity 420. This minimum level of negative pressure may at least equal to the level of negative pressure applied by an expansion force of the one or more compressible members, as described further below. In some embodiments the minimum level of negative pressure is greater than the level of negative pressure applied by the expansion force of the one or more compressible members. In some embodiments the one or more compressible members are configured not to compress until a negative pressure of at least −45 mmHg is applied to the internal cavity 420. In other embodiments the one or more compressible members are configured not to compress until a negative pressure of at least −70 mmHg is applied to the internal cavity 420. In even further embodiments the one or more compressible members are configured not to compress until a negative pressure of at least −200 mmHg is applied to the internal cavity 420.

The compressed one or more compressible members which comprise the transmission layer 430 are thereafter configured to exert an expansion force within the internal cavity 420 that generates a level of negative pressure which may be communicated to the wound. In some embodiments the compression of one or more compressible members results in elastic deformation of the one or more compressible members, and therefore the expansion force exerted by the one or more compressible members corresponds to the stiffness of the one or more compressible members. Preferably, the stiffness of the one or more compressible members is such that upon compression, the compressed members are sufficiently stiff to exert an expansion force that applies a desired level of negative pressure. However, the one or more compressible members are preferably not so stiff that the removal of air from the internal cavity 420 is unable to compress the one or more compressible members. As the expansion force exerted by the one or more compressible members corresponds to the stiffness of the one or more compressible members, the expansion force exerted by the one or more compressible members can be varied by selecting one or more compressible members with an appropriate stiffness.

Again with reference to FIG. 4A, the expansion force generated by the one or more compressible members against the internal cavity 420 applies a negative pressure of at least −45 mmHg to the wound. In other embodiments the expansion force generated by the one or more compressible members against the internal cavity 420 applies a negative pressure of at least −70 mmHg to the wound. In even further embodiments the expansion force generated by the one or more compressible members against the internal cavity 420 applies a negative pressure of between about −20 mmHg to about −200 mmHg to the wound.

The wound dressing 400 as illustrated in FIGS. 4A-C may be used to treat a wound site on a patient. The healthy skin surrounding the wound site is preferably cleaned and excess hair removed or shaved. The wound site may also be irrigated with sterile saline solution if necessary. Optionally, a skin protectant may be applied to the skin surrounding the wound site. If necessary, a wound packing material, such as foam or gauze, may be placed in the wound site. This may be preferable if the wound site is a deeper wound.

After the skin surrounding the wound site is dry the wound dressing 400 may be positioned and placed over the wound site. Preferably, the wound dressing 400 is placed with the wound contact layer 460 over and/or in contact with the wound site. In some embodiments, an adhesive layer is provided on the lower surface of the wound contact layer 460, which may in some cases be protected by an optional release layer to be removed prior to placement of the wound dressing 400 over the wound site. To help ensure adequate sealing for TNP, the edges of the dressing 400 are preferably smoothed over to avoid creases or folds.

Once the wound dressing 400 has been secured over a wound site, the wound dressing 400 can be operated to apply negative pressure to the wound. A sufficient amount of air is removed from the internal cavity 420 of wound dressing 400 by any suitable mechanism as described above. For example, a user may apply even pressure to the top surface of the wound dressing 400 with a hand. The removal of a sufficient amount of air causes the transmission layer 430 comprising one or more compressible members to compress as described above. The compressed one or more compressible members which comprise the transmission layer 430 are thereafter configured to exert an expansion force within the internal cavity 420 that generates a level of negative pressure which is then applied to the wound site.

Treatment of the wound site preferably continues until the wound has reached a desired level of healing. The level of negative pressure at the wound site may diminish over time, which may be due in part to small leaks which may occur in the seal between the wound dressing 400 and the skin surrounding the wound site. As the level of negative pressure at the wound site diminishes, the wound dressing 400 may again be compressed to evacuate air in order to continue to apply a desired level of negative pressure to the wound site. The wound dressing 400 can be operated as described above multiple times to apply a desired level of negative pressure to the wound site, as may be deemed necessary by one of skill in the art. In some embodiments, the negative pressure apparatuses and methods described herein may further comprise a second negative pressure source (such as a miniature pump) that may be incorporated onto or into the wound dressing or compressible negative pressure source as described above. Alternatively, the second negative pressure source may be fluidically connected to either the wound dressing or the compressible negative pressure source. The pump may be a micro pump, or miniature pump, for example the micro pump 110 as described in US 20140249493 or the pump 170 as described in US 20140228791, both of which are hereby incorporated by reference in their entireties. The miniature pump may also be a pump similar to pumps described in U.S. Publication Nos. 20130110058 and 20150100045 and WO 2013/136181, the entireties of each of which are hereby incorporated by reference.

If fluidically connected to the compressible negative pressure source 100 as described in FIGS. 1A-1C, the pump may communicate with the internal cavity 120 via a port, valve, hole, or aperture in the fluid impermeable outer membrane 110. The pump may be used to apply negative pressure to the internal cavity 120 via the aperture. In some embodiments the pump may be integral with the compressible negative pressure source 100. For example, the pump may be mounted on the fluid impermeable outer membrane 110. The pump may be mounted on the fluid impermeable outer membrane 110 using, for example, a pressure sensitive or UV adhesive.

In some other embodiments the pump may be located inside the internal cavity 120 and may further communicate with the ambient environment via a port, valve, hole, or aperture in the fluid impermeable outer membrane 110. The aperture may allow the pump to exhaust air from the internal cavity 120 into the ambient environment to thereby generate negative pressure in the internal cavity 120. In some embodiments the pump may be surrounded by or enclosed in the spacer layer 130. In other embodiments the pump may be disposed above or below the spacer layer 130 in the internal cavity 120.

The pump may be configured to regulate the negative pressure applied to the internal cavity 120 of the compressible negative pressure source 100. That is, in some embodiments the pump is configured to selectively turn on or off so as to regulate the level of negative pressure applied to the internal cavity 120. The pump may be configured to automatically turn on, or activate, in response to the level of negative pressure in the internal cavity 120 rising above a first threshold, and may be configured to selectively turn off, or deactivate, in response to the level of negative pressure in the internal cavity 120 falling below a second threshold. The pump may also be configured to be activated and deactivated manually by a user.

In use, the pump may be activated to remove a sufficient amount of air from the internal cavity 120 of the compressible negative pressure source 100. The removal of a sufficient amount of air causes the spacer layer 130 comprising one or more compressible members to compress as described above. The compressed one or more compressible members which comprise the spacer layer 130 are thereafter configured to exert an expansion force within the internal cavity 120 that generates a level of negative pressure which is then communicated to the wound dressing 310 via the conduit. The negative pressure is then applied to the wound site 301 by the wound dressing 310. When a level of negative pressure in the internal cavity rises above a first threshold the pump may automatically active to remove air from the internal cavity 120 until a desired level of negative pressure has been reached, at which point the pump may automatically turn off. Such a cycle may be repeated multiple times to continue to apply negative pressure to the wound site 301, as may be deemed necessary by one of skill in the art.

The internal cavity 120 may act as a negative pressure reservoir which may serve to increase the amount of time the pump is off. That is, the compressible negative pressure source 100 may allow a desired level of negative pressure to be applied to a desired location, for example a wound dressing 301, for a longer period of time than would be achievable with a pump alone. By reducing the amount of time that a pump may need to run or be turned on to apply a desired level of negative pressure to a desired location the compressible negative pressure source 100 may be less intrusive to the daily life of a user.

Alternatively, a second negative pressure source, for example a miniature pump, may be incorporated into the wound dressing 310 or wound dressing 400 as described above. For example, the second negative pressure source may be mounted to an outer surface of the wound dressing, such as to a top layer of the wound dressing, or may be provided internal to the wound dressing (e.g., underneath a top layer). The second negative pressure source may be configured to provide negative pressure to the wound site. The compressible negative pressure source described above (e.g., negative pressure source 110 or the compressible members of transmission layer 430) may provide an additional mechanism to generate or maintain negative pressure at the wound site, reducing the amount of time that the pump needs to run or be turned on.

In further embodiments, the second negative pressure source is located remote or away from the wound dressing and configured to connect to the wound dressing with one or more fluid conduits. This second negative pressure source may provide a first mechanism for providing negative pressure to the wound site, and a compressible negative pressure source as described above may provide a second mechanism for providing or maintaining negative pressure to the wound site. This may then reduce the amount of time that the second negative pressure source needs to run or be turned on.

Although this disclosure describes certain embodiments, it will be understood by those skilled in the art that many aspects of the methods and devices shown and described in the present disclosure may be differently combined and/or modified to form still further embodiments or acceptable examples. All such modifications and variations are intended to be included herein within the scope of this disclosure. Indeed, a wide variety of designs and approaches are possible and are within the scope of this disclosure. No feature, structure, or step disclosed herein is essential or indispensable. Moreover, while illustrative embodiments have been described herein, the scope of any and all embodiments having equivalent elements, modifications, omissions, combinations (e.g., of aspects across various embodiments), substitutions, adaptations and/or alterations as would be appreciated by those in the art based on the present disclosure. While certain embodiments have been described, these embodiments have been presented by way of example only, and are not intended to limit the scope of protection.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A wound treatment apparatus comprising:
a flexible member configured to apply negative pressure to a wound, comprising:
a fluid impermeable outer membrane defining an internal cavity;
one or more compressible members disposed in said internal cavity, wherein the one or more compressible members are configured to compress upon removal of air from the internal cavity;
wherein after the removal of air from the internal cavity, the one or more compressible members are configured to exert an expansion force within the internal cavity that applies a desired level of negative pressure to the wound.

2. The apparatus of claim 1, wherein the flexible member is a wound dressing further comprising a wound contact layer, wherein the one or more compressible members are disposed in the internal cavity defined between the fluid impermeable outer membrane and the wound contact layer.

3. (canceled)

4. The apparatus of claim 1, wherein the fluid impermeable outer membrane comprises a fluid impermeable upper membrane and a fluid impermeable lower membrane, wherein said upper membrane and said lower membrane are sealed together to form the internal cavity therebetween.

5. The apparatus of claim 1, wherein the fluid impermeable outer membrane comprises a bag defining the internal cavity.

6. The apparatus of claim 1, further comprising a separate wound dressing configured to be positioned onto a wound, wherein the flexible member is configured to be in fluid communication with the separate wound dressing and apply negative pressure to the wound through the separate wound dressing.

7. (canceled)

8. The apparatus of claim 6, further comprising a conduit in fluid communication with the internal cavity of the flexible member and configured to communicate negative pressure generated by the expansion force of the one or more compressible members to the separate wound dressing.

9. The apparatus of claim 8, further comprising a regulator valve for regulating the negative pressure supplied to the wound dressing by the internal cavity of the flexible member via the conduit.

10. (canceled)

11. The apparatus of claim 1, wherein the one or more compressible members comprises a plurality of constant tension springs.

12. The apparatus of claim 1, wherein a 3D knitted or fabric material incorporates a plurality of compressible members.

13. The apparatus of claim 12, wherein the 3D knitted or fabric material comprises a first fabric layer and a second fabric layer and a plurality of spacer elements extending therebetween.

14. The apparatus of claim 12, wherein the 3D knitted or fabric layer comprises a top textured polyester layer, a bottom flat polyester layer and a third fiber layer sandwiched therebetween.

15. The apparatus of claim 1, wherein the one or more compressible members comprise foam.

16. The apparatus of claim 1, wherein the one or more compressible members are configured to exert an expansion force within the internal cavity that applies at least −45 mm Hg to the wound.

17. The apparatus of claim 1, further comprising tubing connected to the flexible member, wherein the tubing provides an air leak to allow air to be removed from the internal cavity.

18. The apparatus of claim 1, further comprising a valve configured to regulate the removal of air from the internal cavity.

19. The apparatus of claim 1, wherein the flexible member is configured to allow removal of air from the internal cavity by squeezing the flexible member.

20. The apparatus of claim 1, further comprising a source of negative pressure configured to remove air from the internal cavity.

21. The apparatus of claim 1, further comprising a port in fluid communication with the internal cavity, wherein the port is configured to communicate with a source of negative pressure.

22. The apparatus of claim 1, further comprising an audible alarm system, wherein the audible alarm system is configured to sound when the pressure in the internal cavity of the flexible member rises above a predetermined negative pressure.

23. A method of generating negative pressure, comprising:
providing a flexible member comprising a fluid impermeable outer membrane defining an internal cavity, and one or more compressible members disposed in the internal cavity; and
removing air from the internal cavity, wherein the removal of air causes the one or more compressible members to compress;
wherein after the removal of air from the internal cavity, the one or more compressible members exert an expansion force within the internal cavity to generate a negative pressure to a desired location.

24.-34. (canceled)
